(19) United States
(12) Patent Application Publication
(10) Pub. No.: US 2024/0382113 A1
(43) Pub. Date: Nov. 21, 2024

(54) METHOD AND SYSTEM FOR PROVIDING AN INTEGRATED ANALYTE SENSOR INSERTION DEVICE AND DATA PROCESSING UNIT

(71) Applicant: ABBOTT DIABETES CARE INC., Alameda, CA (US)

(72) Inventor: Gary Ashley Stafford, Hayward, CA (US)

(21) Appl. No.: 18/657,478

(22) Filed: May 7, 2024

Related U.S. Application Data
(63) Continuation of application No. 16/443,446, filed on Jun. 17, 2019.
Application No. 16/443,446 is a continuation of application No. 15/786,272, filed on Oct. 17, 2017, now Pat. No. 10,362,972.
Application No. 15/786,272 is a continuation-in-part of application No. 14/498,895, filed on Sep. 26, 2014, now Pat. No. 9,808,186.
Application No. 14/498,895 is a continuation of application No. 13/717,501, filed on Dec. 17, 2012, now Pat. No. 8,862,198.
Application No. 13/717,501 is a continuation of application No. 11/530,472, filed on Sep. 10, 2006, now Pat. No. 8,333,714.

(51) Int. Cl. A61B 5/145; A61B 5/00; A61B 5/15; A61M 5/142; A61M 5/172; G16H 10/60; G16H 40/63

(52) U.S. Cl. CPC A61B 5/14503; A61B 5/0002; A61B 5/1411; A61B 5/14514; A61B 5/14532; A61B 5/14546; A61M 5/14244; A61M 5/14276; A61M 5/1723; G16H 10/60; G16H 40/63; A61B 5/150022; A61B 2560/0252; A61M 2005/14208; A61M 2205/502; A61M 2205/82; A61M 2230/201; A61M 2230/50

(57) ABSTRACT

Method and apparatus for providing an integrated analyte sensor and data processing unit assembly is provided.

### RELATED APPLICATIONS

The present application is a continuation of U.S. patent application Ser. No. 16/443,446, filed Jun. 17, 2019, which is a continuation of U.S. patent application Ser. No. 15/786,272, filed Oct. 17, 2017, now U.S. Pat. No. 10,362,972, which is a continuation of U.S. patent application Ser. No. 14/498,895, filed Sep. 26, 2014, now U.S. Pat. No. 9,808,186, which is a continuation of U.S. patent application Ser. No. 13/717,501, filed Dec. 17, 2012, now U.S. Pat. No. 8,862,198, which is a continuation of U.S. patent application Ser. No. 11/530,472, filed Sep. 10, 2006, now U.S. Pat. No. 8,333,714, all of which are incorporated herein by reference in their entireties for all purposes.

### BACKGROUND

Analyte monitoring systems generally include a sensor such as a subcutaneous analyte sensor, at least a portion of which is inserted under the skin for fluid contact with interstitial fluid, for detecting analyte levels such as glucose levels, a transmitter (such as an RF transmitter) in communication with the sensor and configured to receive the sensor signals and to transmit them to a corresponding receiver unit by for example, using RF data transmission protocol. The receiver may be operatively coupled to a glucose monitor that performs glucose related calculations and data analysis.

The transmitter is in signal communication with the sensor. Generally, the sensor is configured to detect and measure the glucose levels of the patient over a predetermined period of time, and the transmitter is configured to transmit data corresponding to or associated with the measured glucose levels over the predetermined period of time for further analysis. To initially deploy the sensor so that the sensor electrodes are in fluid contact with the patient's analyte fluids, a separate deployment mechanism such as a sensor inserter or introducer is used. More specifically, the introducer includes a sharp needle shaped inserter that is configured to pierce through the skin of the patient and substantially concurrently guide the sensor through the patient's skin so as to place at least a portion of the sensor in fluid contact with the target biological fluid of the patient.

The inserter is typically used only during the sensor insertion process, and once the sensor is properly and accurately positioned, the inserter and the introducer are discarded. This requires a level of care as the inserter is sharp and may damage other parts of the patient's skin if not properly handled. Further, since the tip of the inserter has come into fluid contact with the patient's biological fluids, it is important to take particular precautions in the handling of the inserter.

Further, to minimize data errors in the continuous or semi-continuous monitoring system, it is important to properly insert the sensor through the patient's skin and securely retain the sensor during the time that the sensor is configured to detect analyte levels. Additionally, for the period of continuous or semi-continuous monitoring which can include, for example, 3 days, 5 days or 7 days, it is important to have the transmitter in proper signal contact with the analyte sensor so as to minimize the potential errors in the monitored data.

In view of the foregoing, it would be desirable to have method and apparatus for providing simple, easy to handle and accurate sensor introduction and retention mechanism for use in an analyte monitoring system. More specifically, it would be desirable to have method and apparatus that minimizes the number of components which the patient has to handle, and which also reduces the number of required steps to properly and accurately position the analyte sensor in fluid contact with the patient's analytes.

### SUMMARY

An apparatus in accordance with one embodiment of the present disclosure includes a disposable assembly including, a housing, a data processing unit disposed in the housing, an introducer disposed within the housing and including a first portion having a sharp distal end configured for piercing through a skin layer, where the first portion of the introducer is retained within the housing after piercing through the skin layer, and an analyte sensor including a first portion and a second portion, the first portion of the analyte sensor coupled to the first portion of the introducer, and the second portion of the analyte sensor coupled to the data processing unit, where the first portion of the analyte sensor is configured for transcutaneous placement so as to be in fluid contact with an interstitial fluid, where the second portion of the analyte sensor is in electrical contact with the data processing unit prior to transcutaneous placement of the first portion of the analyte sensor, and where the housing, the data processing unit and the introducer are assembled to form the disposable assembly, and the data processing unit includes an aperture through which the introducer is removable.

An integrated assembly in accordance with another embodiment includes a housing, a data processing unit disposed within the housing, an introducer having at least a portion disposed within the housing and including a first portion having a sharp distal end configured for piercing through a skin layer, where the first portion of the introducer is retained within the housing after piercing through the skin layer, and an analyte sensor coupled to the housing, a first portion of the analyte sensor configured for subcutaneous placement and so as to be in fluid contact with interstitial fluid under the skin layer, and a second portion of the analyte sensor disposed within the housing and in electrical communication with the data processing unit prior to subcutaneous placement of the first portion of the analyte sensor, where the housing, the data processing unit, the introducer, and the analyte sensor are assembled as a single disposable unit, and the data processing unit includes an aperture through which the introducer is removable.

In the manner described, within the scope of the present invention, the integrated analyte sensor and data processing unit assembly in accordance with the various embodiments is configured to integrate an analyte sensor, a sensor introducer mechanism, and a data processing device into a single assembly which may be disposable, and which allows for simple and accurate sensor deployment to the desired subcutaneous position, and that may be easily operated using one hand by the user or the patient.

These and other features and advantages of the present invention will be understood upon consideration of the following detailed description of the invention and the accompanying drawings.

### BRIEF DESCRIPTION OF THE DRAWINGS

FIG. 1 illustrates a side view of the overall assembly of an integrated analyte sensor delivery and data processing unit in accordance with one embodiment of the present invention;

FIG. 2 illustrates a side view of the integrated analyte sensor delivery and data processing unit of FIG. 1 without a protective guard in accordance with one embodiment of the present invention;

FIGS. 3A-3C illustrate a perspective view, a top planar view and a bottom planar view, respectively of the integrated analyte sensor delivery and data processing unit of FIG. 1 in accordance with one embodiment of the present invention;

FIG. 4A illustrates the integrated analyte sensor delivery and data processing unit of FIG. 1 with the introducer removed in accordance with one embodiment;

FIG. 4B illustrates the integrated analyte sensor delivery and data processing unit without the introducer in accordance with one embodiment of the present invention;

FIG. 5A illustrates the introducer and the analyte sensor assembly with the protective guard in accordance with one embodiment of the present invention;

FIGS. 5B-5C illustrate a side view and a perspective view, respectively, of the introducer and analyte sensor assembly in accordance with one embodiment of the present invention;

FIG. 6 illustrates a block diagram of an analyte monitoring system with integrated analyte sensor delivery and data processing unit in accordance with one embodiment of the present invention; and

FIG. 7 illustrates a block diagram of the data processing unit of the integrated analyte sensor delivery and data processing unit in accordance with one embodiment of the present invention.

### DETAILED DESCRIPTION

In accordance with the various embodiments of the present invention, there is provided an integrated analyte sensor delivery and data processing unit for subcutaneous placement of an analyte sensor for fluid contact with an analyte of a subject for analyte monitoring, and data processing associated with the monitored analyte levels. More specifically, the integrated analyte sensor delivery and data processing unit in accordance with one embodiment of the present invention provides simple, easy to use analyte sensor placement for analyte monitoring and associated data processing without a direct line of sight visual aid, and also, which may be easily performed without the use of both hands.

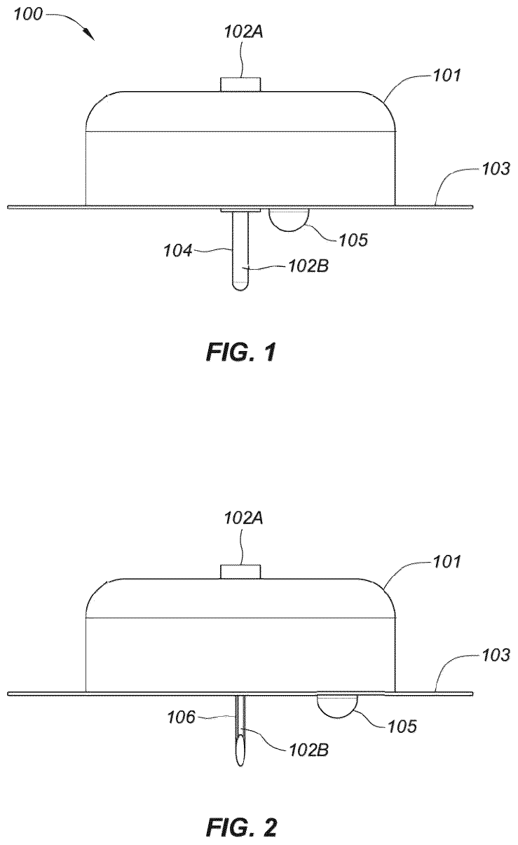

FIG. 1 illustrates a side view of the overall assembly of an integrated analyte sensor delivery and data processing unit in accordance with one embodiment of the present invention. Referring to FIG. 1, in one embodiment, an integrated analyte sensor delivery and data processing unit assembly 100 includes a data processing unit 101 provided on an upper surface of an adhesive patch 103 as shown. The adhesive patch 103 in one embodiment is provided with an adhesive material, such as, for example, polyester or acrylic based adhesives or any other suitable material which are biocompatible providing sufficient adhesive strength, on its bottom surface that is configured to securely attach the adhesive patch 103 and the data processing unit 101 on a surface of a subject such as the skin of a patient.

Referring to FIG. 1, also provided is a temperature module 105 to be in physical contact with the surface of the subject to, among others, detect the temperature, as further discussed in further detail below in conjunction with temperature measurement section 703 of FIG. 7. Referring again to FIG. 1, the integrated sensor delivery and data processing unit assembly 100 in one embodiment also includes an introducer having an upper portion 102A and a lower portion 102B. The upper and lower portions 102A, 102B of the introducer in one embodiment is coupled to the housing of the data processing unit 101 such that the upper portion 102A of the introducer protrudes from an upper surface of the data processing unit 101, while the lower portion 102B of the introducer is configured to protrude downwardly from the bottom surface of the data processing unit 101 as can be seen in FIG. 1.

As shown in FIG. 1, the upper portion 102A of the introducer is configured to guide the user to insert the introducer through the surface of the subject such as the skin of the patient. Thus, the upper portion 102A of the introducer in one embodiment is provided with a larger surface area (that is, for example, a larger diameter) to apply force thereon, while the lower portion 102B of the introducer is configured with a sharp tip to facilitate puncturing or piercing through the surface of the subject such as the skin of the patient.

Additionally, in one aspect, there is provided a guard segment 104 in the integrated analyte sensor and data processing unit assembly 100 such that the guard segment 104 is configured to substantially cover the lower portion 102B of the introducer. In one embodiment, the guard segment 104 is configured as a protective needle guard so as to maintain the lower portion 102B of the introducer in a substantially sterile environment prior to subcutaneous placement through the surface of the subject such as the skin of the patient. Moreover, in one aspect, the guard segment 104 is configured to protect the sharp edge of the lower portion 102B of the introducer from inadvertent contact with the subject, for example, prior to the subcutaneous deployment of the analyte sensor to avoid, for example, contamination of the lower portion 102B of the introducer, or potential injury from the sharp edge of the lower portion 102B of the introducer.

More specifically, FIG. 2 illustrates a side view of the integrated analyte sensor delivery and data processing unit of FIG. 1 without a guard segment in accordance with one embodiment of the present invention. Referring to FIG. 2, with the guard segment 104 of FIG. 1 removed, it can be seen that in one embodiment, analyte sensor 106 is provided in the integrated analyte sensor delivery and data processing unit 100 such that at least a portion of the analyte sensor 106 is disposed within the lower portion 102B of the introducer.

In one embodiment, the patient or the user of the integrated analyte sensor delivery and data processing unit assembly 100 removes the guard segment 104 to expose the lower portion 102B of the introducer, and thereafter, places the entire analyte sensor delivery and data processing unit assembly 100 on the surface of the subject such as the skin layer of the patient with sufficient force applied on the upper surface of the data processing unit 101 such that the lower portion 102B of the introducer is pierced through the skin layer of the patient. Thereafter, the introducer may be removed to detach or decouple from the data processing unit 101 by, for example, pulling at the upper portion 102A of the introducer, thereby withdrawing the introducer from the patient and separating from the data processing unit 101, while retaining the analyte sensor 106 (FIG. 2) in position in fluid contact with the patient's analyte.

Referring again to FIGS. 1 and 2, in one embodiment, the bottom layer of the adhesive patch 103 may be provided with a protective layer (not shown) which the patient or the user of the integrated analyte sensor delivery and data processing unit assembly 100 removes (for example, by peeling off to detach from the bottom surface of the adhesive patch 103 and thus exposing the adhesive material on the bottom surface of the adhesive patch 103) prior to subcutaneously positioning the analyte sensor in the patient. For example, in one embodiment, the guard segment 104 may be first removed and the protective layer removed before use, or alternatively, the removal of the protective layer may be configured to remove or detach the guard segment 104 therewith. In an alternate embodiment, the protective layer and the guard segment 104 may be formed as a single integrated unit for ease of use.

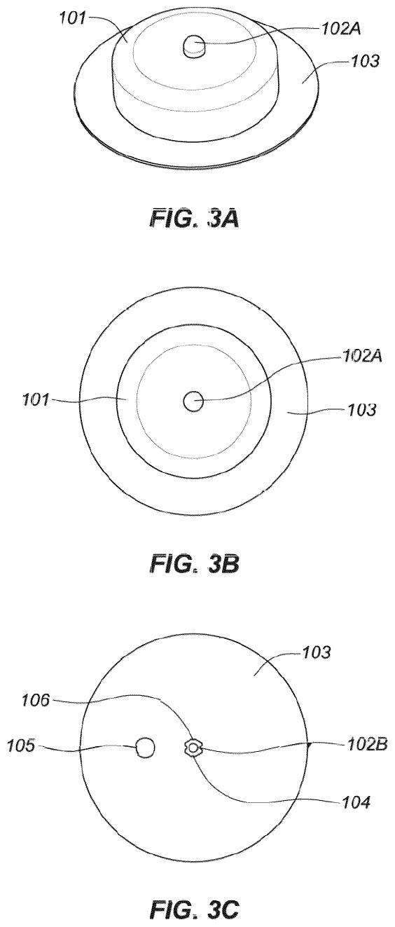

FIGS. 3A-3C illustrate a perspective view, a top planar view and a bottom planar view, respectively of the integrated analyte sensor delivery and data processing unit of FIG. 1 in accordance with one embodiment of the present invention.

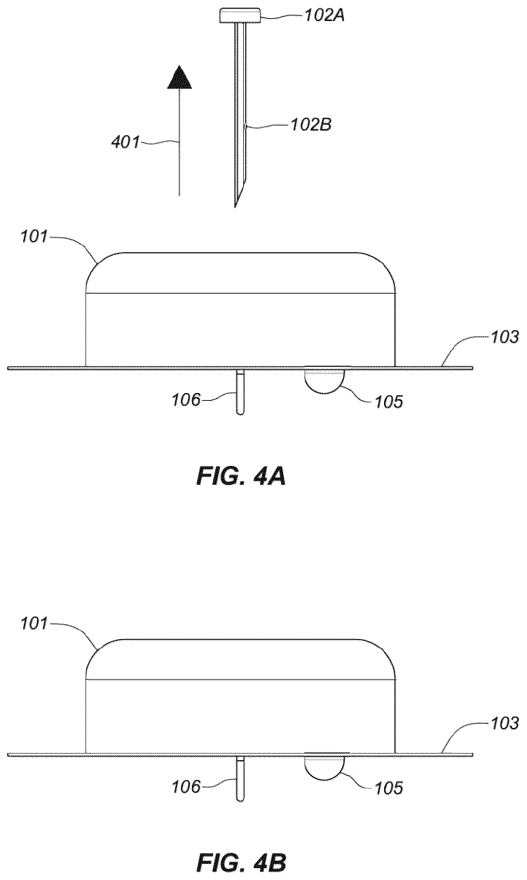

Referring now to FIG. 4A, the removal of the introducer is shown. More specifically, FIG. 4A illustrates the integrated analyte sensor delivery and data processing unit 100 of FIG. 1 with the introducer removed, and FIG. 4B illustrates the integrated analyte sensor delivery and data processing unit without the introducer in accordance with one embodiment of the present invention. That is, in one embodiment, upon placement of the integrated analyte sensor delivery and data processing unit assembly 100 on the skin surface of the patient, for example, the patient retracts or pulls the introducer substantially at the upper portion 102A in the direction substantially perpendicular and away from the data processing unit 101 as shown by the directional arrow 401.

When the introducer is removed, in one embodiment, the entire introducer including the upper portion 102A and the lower portion 102B is withdrawn from the housing of the data processing unit 101 to completely separate from the data processing unit. Moreover, the portion of the analyte sensor 106 is retained in the subcutaneous position so as to maintain fluid contact with the patient's analyte. In one embodiment, the housing of the data processing unit 101 is provided with a self-sealing aperture (not shown) through which the introducer may be removed, such that, when the introducer is withdrawn, there is no opening or aperture on the data processing unit 101 housing where moisture or contaminant may compromise the functions and operations of the data processing unit 101. Optionally, while not shown, a protective layer may be provided over the integrated analyte sensor delivery and data processing unit 100 upon positioning on the skin of the patient to provide protection from water, moisture or any other potential contaminants potentially damaging the integrated analyte sensor delivery and data processing unit 100.

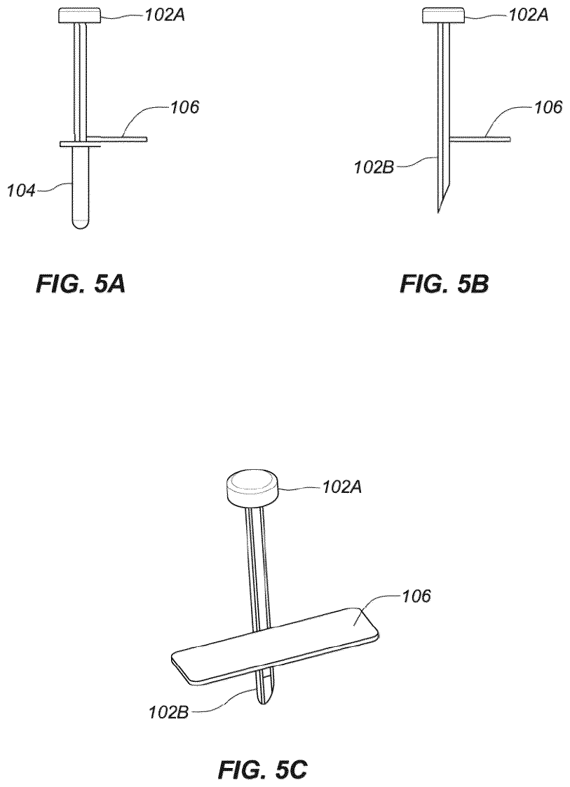

FIG. 5A illustrates the introducer and the analyte sensor assembly with the protective guard, and FIGS. 5B-5C illustrate a side view and a perspective view, respectively, of the introducer and analyte sensor assembly in accordance with one embodiment of the present invention. As shown in the Figures, the integrated analyte sensor delivery and data processing unit 100 in one embodiment may be pre-assembled as a single integrated unit with the analyte sensor 106 in electrical contact with the data processing unit 101, and further, where a portion of the analyte sensor 106 is disposed within the lower portion 102B of the introducer such that, the user or patient may easily, and accurately position the analyte sensor 106 under the skin layer to establish fluid contact with the patient's analyte, and thereafter, to provide the detected analyte levels from the analyte sensor 106 to the data processing unit 101.

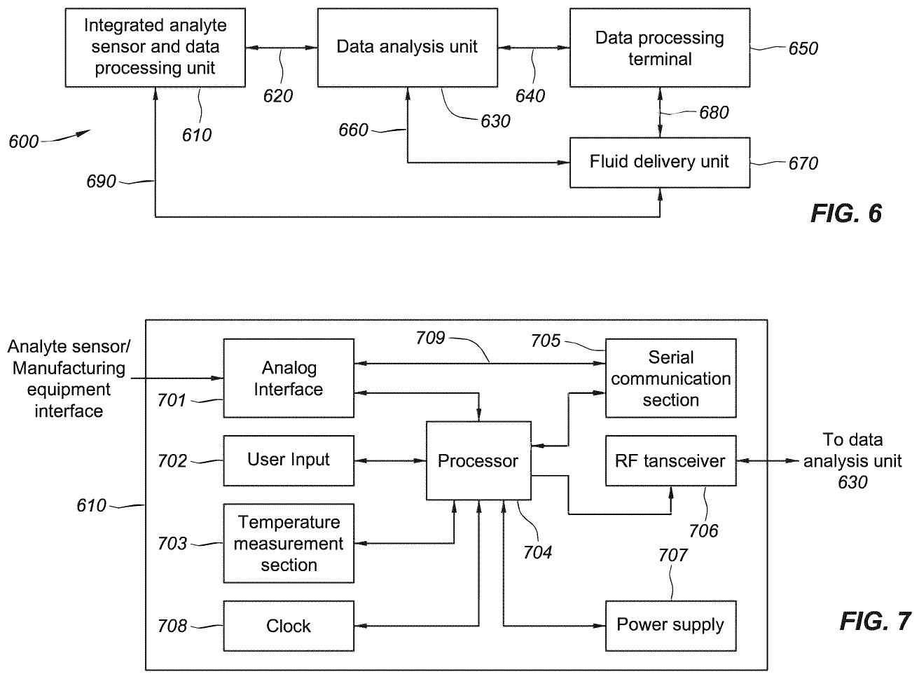

FIG. 6 illustrates a block diagram of an analyte monitoring system with integrated analyte sensor delivery and data processing unit in accordance with one embodiment of the present invention. Referring to FIG. 6, a data monitoring and management system 600 such as, for example, an analyte (e.g., glucose) monitoring and management system in accordance with one embodiment of the present invention is shown. The subject invention is further described primarily with respect to a glucose monitoring system for convenience and such description is in no way intended to limit the scope of the invention. It is to be understood that the analyte monitoring system may be configured to monitor a variety of analytes, e.g., lactate, and the like.

Analytes that may be monitored include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored.

The data monitoring and management system 600 in one embodiment includes an integrated analyte sensor and data processing unit 610, a data analysis unit 630 which is configured to communicate with the integrated analyte sensor and data processing unit 610 via a communication link 620. The data analysis unit 630 may be further configured to transmit and/or receive data to and/or from a data processing terminal 650 via communication link 640. The data processing terminal 650 in one embodiment may be configured for evaluating the data received by the data analysis unit 630.

Referring again to FIG. 6, also shown is a fluid delivery unit 670 which is operatively coupled to the data processing terminal 650 via communication link 680, and further operatively coupled to the data analysis unit 630 via communication link 660, and also, operatively coupled to the integrated analyte sensor and data processing unit 610 via communication link 690. In one embodiment, the fluid delivery unit 670 may include an external or implantable infusion device such as an insulin infusion pump, or the like, which may be configured to administer insulin to patients, and which may be configured to determine suitable modifications or updates to the medication dispensing profile based on data received from one or more of the integrated analyte sensor and data processing unit 610, data analysis unit 630, or data processing terminal 650, for example, for administering and modifying basal profiles, as well as for determining appropriate boluses for administration based on, among others, the detected analyte levels received from the integrated analyte sensor and data processing unit 610.

Furthermore, referring again to FIG. 6, the one or more of the communication links 620, 640, 660, 680, and 690 may be configured as one or more of a wired or a wireless communication link, for example, including but not limited to RS232 cable connection, a Universal Serial Bus (USB) connection, an RF communication link, an infrared communication link, a Bluetooth® enabled communication link, an 802.11x wireless communication link, or an equivalent wireless communication protocol which would allow secure, wireless communication of several units (for example, per HIPAA requirements) while avoiding potential data collision and interference.

Moreover, it will be appreciated by one of ordinary skill in the art that the data monitoring and management system 600 may include one or more integrated analyte sensor and data processing unit 610, one or more data analysis unit 630, one or more fluid delivery unit 670 and one or more data processing terminal 650. In addition, the one or more integrated analyte sensor and data processing unit 610, one or more data analysis unit 630, one or more fluid delivery unit 670 and one or more data processing terminal 650 may be in communication with a remote site over a data network such as the internet for transmitting and/or receiving information associated with the functions and operations of each device. For example, the one or more integrated analyte sensor and data processing unit 610, one or more data analysis unit 630, one or more fluid delivery unit 670 and one or more data processing terminal 650 may be in communication with a data network such as the internet for retrieving and/or transmitting data from a remote server terminal.

Furthermore, in one embodiment, in a multi-component environment, each device is configured to be uniquely identified by each of the other devices in the system so that communication conflict is readily resolved between the various components within the data monitoring and management system 100 of FIG. 1.

In one embodiment of the present invention, the sensor 106 of FIG. 2 is physically positioned in or on the body of a user whose analyte level is being monitored. The sensor 106 may be configured to continuously sample the analyte level of the user and convert the sampled analyte level into a corresponding data signal for transmission by the data processing unit 101 of FIG. 1. More specifically, in one embodiment, the data processing unit 101 may be configured to perform data processing such as filtering and encoding of data signals, each of which corresponds to a sampled analyte level of the user, for transmission to the data analysis unit 630 via the communication link 620.

In one embodiment, the communication link 620 may be configured as a one-way RF communication path from the integrated analyte sensor and data processing unit 610 to the data analysis unit 630. In such embodiment, the data processing unit 101 of the integrated analyte sensor and data processing unit 610 is configured to transmit the sampled data signals received from the sensor 106 without acknowledgement from the data analysis unit 630 that the transmitted sampled data signals have been received. For example, the data processing unit 101 may be configured to transmit the encoded sampled data signals at a fixed rate (e.g., at one minute intervals) after the completion of the initial power on procedure. Likewise, the data analysis unit 630 may be configured to detect the encoded sampled data signals transmitted from the data processing unit 101 at predetermined time intervals. Alternatively, the communication link 620 may be configured with a bi-directional RF (or otherwise) communication between the data processing unit 101 and the data analysis unit 630.

Referring again to FIG. 6, in one embodiment, the data processing terminal 650 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 650 may further be connected to a remote data network such as over the internet (not shown) for storing, retrieving and updating data corresponding to the detected analyte level of the user and/or therapy related information such as medication delivery profiles prescribed by a physician, for example.

FIG. 7 illustrates a block diagram of the data processing unit of the integrated analyte sensor delivery and data processing unit 610 in accordance with one embodiment of the present invention. Referring to FIGS. 6 and 7, the data processing unit 610 in one embodiment includes an analog interface 701 configured to communicate with the sensor 106 (FIG. 2), a user input 702, and a temperature detection section 703, each of which is operatively coupled to a data processing unit processor 704 such as one or more central processing units (CPUs) or equivalent microprocessor units.

Further shown in FIG. 7 are a transmitter serial communication section 705 and an RF transceiver 706, each of which is also operatively coupled to the processor 704. In one embodiment, the serial communication section 705 may be operatively coupled to the analog interface 701 via communication link 709. Moreover, a power supply 707 such as a battery is also provided in the data processing unit 610 to provide the necessary power for the components in the data processing unit 610. Additionally, as can be seen from the figure, clock 708 is provided to, among others, supply real time information to the processor 704.

Referring back to FIG. 7, the power supply section 707 in one embodiment may include a rechargeable battery unit that may be recharged by a separate power supply recharging unit (for example, provided in the data analysis unit 630 (FIG. 6)) so that the data processing unit 101 may be powered for a longer period of usage time. In addition, the temperature measurement (or detection) section 703 of the data processing unit 610 is configured to monitor the temperature of the skin near the sensor insertion site. The temperature reading may be used to adjust the analyte readings obtained from the analog interface 701.

In this manner, in one embodiment, the sensor detected analyte levels are provided to the data processing unit of the integrated analyte sensor and data processing unit 610 (FIG. 6), for example, as current signals, and which are in turn, converted to respective digital signals for transmission (including, for example, RF transmission) to the data analysis unit 630, fluid delivery unit 670, and/or the data processing terminal 650 of FIG. 6 for further processing and analysis (including drug (e.g., insulin) therapy management, infusion control, and health monitoring and treatment, for example). That is, the monitored analyte data may be used by the patient and/or the patient's healthcare provider to modify the patient's therapy such as an infusion protocol (such as basal profile modifications in the case of diabetics) as necessary to improve insulin infusion therapy for diabetics, and further, to analyze trends in analyte levels for improved treatment.

Additional detailed description of the data monitoring and management system such as analyte monitoring systems, its various components including the functional descriptions of data processing unit and data analysis unit are provided in U.S. Pat. No. 6,175,752 issued Jan. 16, 2001 entitled “Analyte Monitoring Device and Methods of Use”, and in U.S. Pat. No. 7,811,231 issued Oct. 12, 2010 entitled “Continuous Glucose Monitoring System and Methods of Use”, each assigned to the Assignee of the present application.

In the manner described above, in one embodiment, the integrated analyte sensor and data processing unit assembly is configured to integrate an analyte sensor, a sensor introducer mechanism, and a data processing device into a single disposable assembly which allows for simple and accurate sensor deployment to the desired subcutaneous position, and which may be used with one hand by the user or the patient. Accordingly, a separate sensor introducing device such as a separate insertion gun or a separate sensor delivery mechanism is not necessary.

Furthermore, by integrating the analyte sensor, the introducer as well as the data processing unit into a single assembly, it is possible to have a smaller profile, simpler use application with less packaging thereby achieving cost reduction in manufacturing. Indeed, by reducing the number of components needed for sensor placement, within the scope of the present invention, other benefits such as reduction in material cost, weight, packaging, and associated handling and disposal may be achieved.

An apparatus including an analyte sensor and a data processing unit in accordance with one embodiment of the present invention includes a housing, a data processing unit coupled to the housing, an introducer removably coupled to the housing, the introducer including a first portion configured for piercing through a skin layer of a subject, and an analyte sensor coupled to the housing, the analyte sensor including a first portion and a second portion, the first portion of the analyte sensor coupled to the first portion of the introducer, and the second portion of the analyte sensor coupled to the data processing unit, where the first portion of the analyte sensor is configured for transcutaneous placement so as to be in fluid contact with an interstitial fluid of the subject, where the second portion of the analyte sensor is in electrical contact with the data processing unit, and further, where at least a portion of the data processing unit, at least a portion of the introducer and at least a portion of the analyte sensor are coupled to the housing as a single integrated assembly.

In one embodiment, there is further provided an adhesive layer substantially on a lower surface of the housing, the adhesive layer configured to removably attach the housing to the skin layer of the subject.

Additionally, a guard segment may be removably coupled to the first portion of the introducer, where the guard segment may be configured to substantially seal the first portion of the introducer.

In a further aspect, the first portion of the introducer may include a sharp tip for piercing through the skin layer of the subject, wherein when the sharp tip is pierced through the skin layer, the first portion of the analyte sensor is transcutaneously placed under the skin layer of the subject so as to be in fluid contact with the interstitial fluid of the subject.

In another aspect, the introducer may be configured to decouple from the housing after the first portion of the analyte sensor is transcutaneously positioned under the skin layer of the subject.

The analyte sensor may include a glucose sensor.

The data processing unit in one embodiment may include a data transmission unit configured to receive one or more signals associated with an analyte level of the subject from the analyte sensor, where the data transmission unit may be configured to wirelessly transmit data associated with the one or more signals received from the analyte sensor, where the data transmission unit may include an RF data transmission unit.

An integrated assembly in accordance with another embodiment of the present invention includes a housing, a data processing unit substantially disposed within the housing, an introducer removably coupled to the housing, at least a portion of the introducer disposed within the housing, and an analyte sensor coupled to the housing, a first portion of the analyte sensor configured for subcutaneous placement and in fluid contact with an interstitial fluid of a subject, and a second portion of the analyte sensor disposed within the housing and in electrical communication with the data processing unit.

The second portion of the analyte sensor in one embodiment may be permanently coupled to the data processing unit.

In another aspect, the introducer may be configured to decouple from the housing after the second portion of the analyte sensor is transcutaneously positioned under the skin layer of the subject.

An insertion kit in accordance with still another embodiment includes a housing, a data processing unit substantially disposed within the housing, an introducer removably coupled to the housing, at least a portion of the introducer disposed within the housing, and an analyte sensor coupled to the housing, a first portion of the analyte sensor configured for subcutaneous placement and in fluid contact with an interstitial fluid of a subject, and a second portion of the analyte sensor disposed within the housing and in electrical communication with the data processing unit.

In one embodiment, the introducer may be manually removed from the housing.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1-18. (canceled)

19. An apparatus, comprising:
a housing;
a data processing unit disposed in the housing;
an introducer disposed within the housing and including a portion having a sharp distal end configured for piercing through a body of a subject, wherein the portion of the introducer is removed from the housing after piercing the body of the subject;
an analyte sensor including a first portion and a second portion, the first portion of the analyte sensor at least partially disposed, prior to removal of the introducer, within the portion of the introducer having the sharp distal end, and the second portion of the analyte sensor coupled to the data processing unit; and
a guard segment for the analyte sensor and the introducer;
wherein the first portion of the analyte sensor is configured for positioning in fluid contact with a biological fluid in the subject, and the second portion of the analyte sensor is in electrical contact with the data processing unit prior to positioning of the first portion of the analyte sensor.

20. The apparatus of claim 19, further including an adhesive layer provided on a lower surface of the housing, wherein the adhesive layer is configured to removably attach the housing to the body of the subject.

21. The apparatus of claim 20, wherein the adhesive layer includes a removable protective layer.

22. The apparatus of claim 19, wherein the apparatus is configured such that when the sharp distal end pierces the body of the subject, the first portion of the analyte sensor is positioned in fluid contact with the biological fluid in the subject.

23. The apparatus of claim 19, wherein the introducer is configured to decouple from the data processing unit after the first portion of the analyte sensor is positioned in the body of the subject.

24. The apparatus of claim 19, wherein the data processing unit includes a data communication unit configured to receive one or more signals associated with a monitored analyte level from the analyte sensor.

25. The apparatus of claim 24, wherein the data communication unit is configured to wirelessly communicate data associated with the one or more signals received from the analyte sensor to a remote location.

26. The apparatus of claim 19, wherein the introducer is positioned through an aperture of the housing.

27. The apparatus of claim 19, wherein the second portion of the analyte sensor extends substantially transverse to the first portion of the analyte sensor.

28. An assembly, comprising:
a housing;
a data processing unit disposed within the housing;
an introducer disposed within the housing and including a portion having a sharp distal end configured for piercing a body of a subject, wherein the portion of the introducer is removable from the housing after piercing the body of the subject;
an analyte sensor coupled to the housing, a first portion of the analyte sensor configured for positioning in fluid contact with a biological fluid in the subject, and a second portion of the analyte sensor disposed within the housing and in electrical communication with the data processing unit prior to positioning of the first portion of the analyte sensor; and
a guard segment for the analyte sensor and the introducer;
wherein the first portion of the analyte sensor is at least partially disposed, prior to removal of the introducer, within the portion of the introducer having the sharp distal end; and
wherein the housing includes an aperture through which the introducer is removable.

29. The assembly of claim 28, wherein the second portion of the analyte sensor is permanently coupled to the data processing unit.

30. The assembly of claim 28, wherein the introducer is configured to decouple from the data processing unit after the first portion of the analyte sensor is positioned in the body of the subject.

31. The assembly of claim 28, wherein the data processing unit includes a data communication unit configured to receive one or more signals associated with a monitored analyte level from the analyte sensor.

32. The assembly of claim 31, wherein the data communication unit is configured to wirelessly transmit data associated with the one or more signals received from the analyte sensor.

33. The assembly of claim 28, further including an adhesive layer provided on a lower surface of the housing, wherein the adhesive layer is configured to removably attach the housing to the body of the subject.

34. The assembly of claim 28, wherein the introducer is positioned through the aperture of the housing.

35. The assembly of claim 28, wherein an opening in the data processing unit is configured to receive the portion of the introducer.

36. The assembly of claim 28, wherein the introducer further includes a proximal portion configured for engaging an outer surface of the data processing unit.
